(19) United States
(12) Patent Application Publication
(10) Pub. No.: US 2012/0149645 A1
(43) Pub. Date: Jun. 14, 2012

(54) COMPOSITIONS AND MODULATION OF MYOCYTE ENHANCER FACTOR 2 (MEF2)

(75) Inventor: Zixu Mao, Atlanta, GA (US)

(73) Assignee: EMORY UNIVERSITY, Atlanta, GA (US)

(21) Appl. No.: 13/323,509

(22) Filed: Dec. 12, 2011

Related U.S. Application Data
(60) Provisional application No. 61/422,306, filed on Dec. 13, 2010.

(51) Int. Cl. A61K 38/00; C12Q 1/68; G01N 33/559; C07K 14/435; A61P 25/16; G01N 33/53; C12Q 1/02

(52) U.S. Cl. 514/17.7; 435/7.2; 435/6.12; 435/6.18; 435/29; 530/350; 514/1.1; 204/456

(57) ABSTRACT

The disclosure relates to mitochondrial myocyte enhancer factor 2 (MEF2), Parkinson's disease, and other related diseases. In certain embodiments, the disclosure relates to analyzing the levels of mitochondrial MEF2 isoforms and/or its mitochondrial target gene ND6 in peripheral blood cells such as white blood cells as an indicator for neuronal mitochondrial MEF2 or ND6 and correlated the level to disease diagnosis, treatment, and prognosis.

### CROSS-REFERENCE TO RELATED APPLICATION

This application claims priority to U.S. Provisional Application No. 61/422,306, filed Dec. 13, 2010, the disclosure of which is incorporated herein in its entirety.

### ACKNOWLEDGEMENTS

This invention was made with government support under Grants AG023695 and NS048254 awarded by the National Institutes of Health. The government has certain rights in the invention.

### FIELD

This disclosure relates to methods of diagnosis and treatment of certain disorders, in particular Parkinson's Disease and disorders involving dysfunction of mitochondrial signaling or protein synthesis.

### BACKGROUND

Mitochondria are the primary energy-generating organelles in most eukaryotic cells. In addition, they also participate in metabolism, calcium signaling, and apoptosis. Mitochondrial dysfunction and the ensuing oxidative stress cause damages to key cellular macromolecules including DNA, which affect many basic biological processes ranging from bioenergetics, gene transcription, to structural integrity. These detrimental effects have been proposed to play important roles in aging, metabolic disorders, and particularly neurodegeneration. Strong evidence from molecular studies, genetics, and mouse models shows that etiological factors associated with Alzheimer's disease, Parkinson's disease, Huntington's disease, amyotrophic lateral sclerosis, hereditary spastic paraplegia, and cerebellar degenerations lead to mitochondrial impairment and may contribute to the pathogenesis of these disorders. Therapeutic approaches targeting mitochondrial dysfunction have great promise; thus, there is a need to identify improved methods of diagnosis and treatment.

Mitochondrial DNA encodes some of the components of enzymatic complexes for oxidative phosphorylation. Proper assembly of a functional oxidative phosphorylation system requires coordination of mitochondrial and nuclear gene expression. The individual strands of the super-coiled circular mtDNA are denoted heavy (H) strand and light (L) strand based on their different buoyant densities. Of the thirteen proteins determined by mtDNA, only one polypeptide ND6 (NADH dehydrogenase 6), a component of complex I, is encoded by the L strand. Mutations in the ND6 gene or alteration in its protein level have been linked etiologically to Leber's Hereditary Optic Neuropathy (LHON) and associated with Parkinson's disease (PD). The basic mtDNA transcription machinery consists of one mitochondrial RNA polymerase and three transcription factors TFAM, TFB1M and TFB2M. They bind to the D-loop promoters in mtDNA to stimulate transcription. However, whether there are mechanisms that control strand specific transcription of mtDNA and how they may be dysregulated under pathological stress is unknown.

Various isoforms of transcription factor MEF2 (MEF2A-D) constitute a group of nuclear proteins found to play important roles in increasing types of cells. For example, MEF2s have been shown to regulate immune cell response, control glucose metabolism in adipocyte, participate in angiogenesis, promote liver fibrosis, and modulate muscle cell differentiation. Many key signaling mechanisms converge on MEF2 to regulate its activity. In neurons, MEF2s are required to regulate neuronal development, synaptic plasticity, as well as survival. Indeed, MEF2s promote the survival of several types of neurons under different conditions. In cellular models, negative regulation of MEF2s by stress and toxic signals contributes to neuronal death. In contrast, enhancing MEF2 activity not only protects primary neurons from death but also attenuates the loss of dopaminergic neurons in substantia nigra pars compacta (SNpc) in a 1-methyl 4-phenyl 1,2,3,6-tetrahydropyridine (MPTP) mouse model of PD. MEF2s may exert their effects by directly regulating the expression of nuclear target genes.

MEF2s are involved in mitochondrial biogenesis. Naya et al., discloses that mice lacking the MEF2A transcription factor have mitochondrial deficiencies. See Naya et al., Nat Med, 2002, 8:1303-1309.

### SUMMARY

The disclosure relates to methods of identifying a subject who has or is at risk of developing Parkinson's disease (PD), and other related diseases. In certain embodiments, a method of diagnosing a disease related to mitochondrial dysfunction in a subject is provided including the steps of analyzing a sample for levels of mitochondrial myocyte enhancer factor 2 (MEF2) isoform and/or its mitochondrial target gene ND6, wherein low levels of the MEF2 isoform and/or ND6 indicate a disease in the subject. In further embodiments, a kit is provided including reagents that provide an indication of MEF2 isoform and/or ND6 levels. In certain instances, the reagents will provide an output when levels of an MEF2 isoform are below a threshold value.

In certain embodiments, the disclosure relates to methods of diagnosing Parkinson's disease or related disease in a subject comprising analyzing a sample for mitochondrial MEF2 isoform levels and correlating low levels of MEF2 isoform to Parkinson's disease or a related disease in the subject. Typically the MEF2 isoform is MEF2A, MEF2B, MEF2C, and/or MEF2D. In certain embodiments, the method further comprises the step of analyzing the sample for cytoplasmic MEF2 and correlating high levels of MEF2 to Parkinson's disease or a related disease in the subject. In certain embodiments, the disclosure relates to methods of diagnosing Parkinson's disease or related disease in a subject comprising analyzing a sample for mitochondrial MEF2D and correlating low levels of MEF2D to Parkinson's disease or a related disease in the subject. In certain other embodiments, the method further comprises the step of analyzing the sample for cytoplasmic MEF2D and correlating high levels of MEF2D to Parkinson's disease or a related disease in the subject.

Generally, the MEF2 isoforms are mitochondrial MEF2A, MEF2B, MEF2C, and/or MEF2D. The isoform levels can be analyzed in a sample that is in peripheral blood, wherein the sample includes peripheral blood cells and white blood cells. In other embodiments, the sample is a neuronal sample and mitochondrial MEF2A, MEF2B, MEF2C, and/or MEF2D levels are measured in this sample and correlated the levels to mitochondrial impairment, disease diagnosis, treatment, and/or prognosis.

In typical embodiments, the disease is Alzheimer's disease, Parkinson's disease, Huntington's disease, amyotrophic lateral sclerosis, hereditary spastic paraplegia, cerebellar degenerations, diabetes mellitus, deafness, Leber's hereditary optic neuropathy, neuropathy, ataxia, retinitis pigmentosa, ptosis, myoneurogenic gastrointestinal encephalopathy, or myoclonic epilepsy with ragged red fibers, or the subject has mitochondrial myopathy, encephalomyopathy, lactic acidosis, or stroke-like symptoms.

In certain embodiments, the disclosure relates to directly enhancing mitochondrial MEF2A, MEF2B, MEF2C, and/or MEF2D levels as a method of treating related diseases. In certain embodiments, the disclosure relates to targeted delivery of MEF2 isoforms specifically to mitochondria to enhance mitochondrial function in therapy. MEF2 mutants have been developed that can be targeted specifically to mitochondria instead of the cell nucleus. Depending on the form of MEF2 (active or dominant negative), it can either block or enhance endogenous mitochondrial MEF2 function. MitoMEF2 (Mt2Dwt or Mt2Ddn) can be used therapeutically to modulate mitochondrial function in cells. In certain embodiments, the disclosure relates to pharmaceutical compositions comprising MEF2 isoforms and mutant forms and methods of treating or preventing related diseases by administering the pharmaceutical composition comprising a MEF2 isoform to a subject at risk of, exhibiting symptoms of, or diagnosed with the disease.

In certain embodiments, the disclosure relates to analyzing a sample for MEF2 isoform levels such as, MEF2A, MEF2B, MEF2C, and/or MEF2D, and correlating aberrant expression to the presence of related diseases.

In certain embodiments, the disclosure relates to drug screening assays, such as, assays that measure normal and/or aberrant levels of MEF2 isoforms by mixing MEF2A, MEF2B, MEF2C, and/or MEF2D with a compound and measuring the effect the compound has on MEF2 isoform activity or transferability to cellular components such as the nucleus or mitochondria.

In certain embodiment, the disclosure relates to isolated chimeric proteins comprising 1) a MEF2 isoform such as MEF2A, MEF2B, MEF2C, and/or MEF2D sequence and 2) a mitochondrial-targeting signal. Typically, the MEF2 sequence lacks a nuclear localization signaling sequence and the second mitochondrial-targeting signal is conjugated to the N-terminus of the MEF2 sequence. In certain embodiments, the MEF2 sequence comprises a mutation. The 30 N-terminal amino acids of wild-type MEF2D contain a mitochondrial targeting signal. This is the endogenous signal of wild-type MEF2D. It is contemplated that a chimeric protein contains an extra targeting signal at the N-terminus, i.e. the endogenous targeting signal and a second targeting signal. In certain embodiments, the disclosure relates to isolated chimeric proteins comprising SEQ ID NO: 2 wherein said isolated chimeric protein does not contain a TAD, MADS box and MEF2 domain, and/or Holliday junction regulator protein family C-terminal repeat. In certain embodiments, the disclosure relates to nucleic acids that encode chimeric proteins disclosed herein. In certain embodiments, the disclosure relates to recombinant vectors such adenoviruses, adeno-associated viruses, plasmids, and lentiviruses that encode nucleic acids that transcribe chimeric proteins disclosed herein.

In some embodiments, the disclosure relates to an isolated chimeric protein comprising, 1) a MEF2D sequence and 2) a second mitochondrial-targeting signal wherein the MEF2D sequence lacks a nuclear localization signaling sequence. In a typically embodiment, the second mitochondrial-targeting signal is conjugated to the N-terminus of the MEF2D sequence which is the 30 N-terminal amino acid residues of MEF2D. In certain embodiments, the MEF2D sequence comprises mutation. In certain embodiments, the disclosure relates to methods of treating Parkinson's Disease or related disease comprising administering a pharmaceutical composition comprising a protein comprising a MEF2D sequence to a subject in need thereof. Typically, the protein is a chimeric protein comprising, 1) a ME2D sequence and 2) a second mitochondrial-targeting signal, wherein said protein is lacking a nuclear localization signaling sequence.

In certain embodiments, the disclosure relates to methods of diagnosing Parkinson's disease or related disease in a subject comprising analyzing a sample for MEF2 mitochondrial target gene ND6 levels and correlating levels of ND6 to Parkinson's disease or a related disease in the subject. In certain embodiments, the disclosure relates to methods of treating diseases related to mitochondrial dysfunction by enhancing ND6 function or expression. In certain embodiments, the molecule is ND6 gene or ND6 protein. In a typical embodiment, the gene or protein is in or expressed in a recombinant vector. In certain embodiments, the disclosure relates to isolated chimeric proteins comprising ND6 and a mitochondrial-targeting signal or nucleic acids that encode such proteins. In certain embodiments, the disclosure relates to methods of treating diseases related to mitochondrial dysfunction comprising administering a pharmaceutical composition comprising a molecule that inhibits or enhances ND6 or ND6 gene expression. In certain embodiments, the molecule is a small molecule agonist or antagonist of ND6, ND6 antibody, aptamer, or siRNA of the ND6 gene.

### BRIEF DESCRIPTION OF THE FIGURES

FIG. 1 shows data on the localization of MEF2D in Mitochondria of Neuronal Cells. (A) Localization of MEF2D in the mitochondrial fraction of a clonal substantia nigral dopaminergic (DA) cell line SN4741 as shown by western blotting (n=3). Cytochrome c (Cyto c) and voltage-dependent anion channel (VDAC) are mitochondrial (Mt) markers; cellular homologue of the v-raf (c-Raf) and glyceraldehydes-3-phosphate dehydrogenase (GAPDH) are cytoplasmic (Cy) markers; poly (ADP-ribose) polymerase (PARP) and histone H1 (H1) are nuclear markers; Glucose-regulated protein 78 kDa (GRP78) is a marker for endoplasmic reticulum. (B) Colocalization of MEF2D (green) with mitochondrial marker MitoTracker (red) in both SN4741 cells (upper panels; scale bar, 10 μm) and primary rat midbrain DA neurons (lower panels; scale bar, 2.5 μm) revealed by immunocytochemistry (n=4). TH, tyrosine hydroxylase, is a marker for dopaminergic neurons. (C) Localization of MEF2D in rat brain mitochondria revealed by immunogold transmission electron microscope (TEM). The control was without primary antibody (n=4; scale bar, 100 nm). (D) Localization of MEF2D in mitochondria of SN4741 cells revealed by immunogold transmission electron microscope (TEM) (n=4; scale bar, 50 nm). (E) Localization of MEF2D in the inner membrane fraction of rat brain mitochondria. Purified rat brain mitochondria were subjected to sub-fractionation and western blotting (n=3). Translocase of the outer membrane 20 kDa (Tom20), Cyto c, complex I 39 kDa protein (39 kDa) and manganese superoxide dismutase (MnSOD) are markers for mitochondrial outer membrane (Om), inter-membrane space (IMS), inner membrane (Im) and matrix (Ma), respectively. (F) In vitro mitochondrial import of MEF2D. Autoradiograph of in vitro translated ³⁵S-MEF2D was shown (n=3). Lane 1: MEF2D control (translated in vitro, 1/10 input); 2: Imported MEF2D (translated in vitro) after incubation with intact, energized mitochondria; 3: Valinomycin (20 μM)-induced loss of membrane potential (ΔΨm) on MEF2D import; 4: Resistance to proteinase K digestion after MEF2D import; 5: Complete digestion of MEF2D by proteinase K after solubilization of mitochondria with Triton X-100.

FIG. 2 shows data on specific sequence and chaperone protein required for localization of MEF2D to mitochondria. (A) Lack of mitochondrial localization by ΔN30MEF2D. Western blotting showed the presence of over-expressed ΔN30MEF2D in both Cy and Nu fractions but not in Mt fraction of SN4741 cells (n=3). VDAC, PARP and c-Raf are Mt, Nu and Cy markers, respectively. Control indicates the control vector group. (B) Immunocytochemistry analysis of mitochondrial localization of transfected Flag-MEF2D. Over-expressed ΔN30MEF2D did not co-localize with MitoTracker in SN4741 cells (n=50 cells; **p<0.01; scale bar, 15 μm). Experiments were repeated four times. (C, D) Requirement of mtHsp70 for mitochondria-targeting of MEF2D (n=4; **p<0.01). Control indicates untreated. The control indicated untreated group. Knocking down mouse mtHsp70 by siRNA reduced MEF2D level in purified mitochondria from SN4741 cells (C). Knocking down mouse mtHsp70 by siRNA did not reduce whole cell MEF2D level in SN4741 cells (D). MnSOD is a known mtHsp70-imported mitochondrial matrix protein.

FIG. 3 shows data on the identification of MEF2D regulatory target in mitochondrial DNA. (A) Presence of a conserved MEF2 consensus site in ND6 gene in mtDNA of different species. Underlined sequence indicates the MEF2 consensus site. Black-shaded areas show the conversed MEF2 site and sequences around it in ND6 gene. (B) Specific binding of MEF2D to the consensus site in ND6 in vitro (n=3). EMSA assay revealed that MEF2D bound to wild type (WT) but not mutant (mt) probe. Arrow indicates the specific binding complex. GST: glutathione S-transferase; GST-MEF2D (1-91): GST-fused MEF2D1-91aa. Hot and cold indicate labeled or unlabeled probe, respectively. (C) Binding of MEF2D to the consensus site in ND6 in cells in vivo (n=3). ChIP assay showed that MEF2D binds to ND6 in SN4741 cells. A fragment bound by anti-MEF2D antibody could only be specifically amplified by PCR with ND6 primers after immuno-precipitation. TFAM, a known mtDNA D-loop binding protein, was used as a control. (D) Sequence analysis of the purified PCR fragment bound by anti-MEF2D antibody in (C) confirmed that it is the predicted ND6 fragment and contains the MEF2 consensus sequence.

FIG. 4 shows data on regulation of mitochondrial gene ND6 by mitochondrial MEF2D. (A) Mitochondria-targeting MEF2D (Mt2D) and mitochondria-targeting dominant-negative MEF2D (Mt2Ddn), which lacks the transactivation domain (TAD). (B) Effects of Mt2D and Mt2Ddn on binding of full length MEF2D to ND6 gene in mitochondria of SN4741 cells revealed by ChIP assay (n=4, **p<0.01). Control indicates the control vector group. (C) Effects of Mt2D or Mt2Ddn on ND6 expression in SN4741 cells. Over-expression of Mt2Ddn in SN4741 cells reduced ND6 but not PPAR-γ coactivator 1 (PGC1) expression by western blotting (n=4, **p<0.01). Control indicates the control vector group. (D) Effects of over-expression of Mt2D or Mt2Ddn on mRNA levels of mitochondria encoded genes. Real-time PCR results showed specific reduction of ND6 mRNA level by over-expression of Mt2Ddn in SN4741 cells (n=4; **P<0.01). Control indicates the control vector group. (E) Effects of Mt2D or Mt2Ddn on mtDNA L-strand transcription initiation in vitro (n=3; **P<0.01). A human mtDNA fragment containing mitochondrial L-strand promoter (LSP) and ND6 gene was used in the in vitro transcription assay. Blot shows ³²P-UTP-labeled transcripts. (F) Effects of Mt2D or Mt2Ddn on mtDNA de novo transcription in vivo (n=3; **P<0.01). Control (−) is without primers. Control (+) indicates the control vector group.

FIG. 5 shows data on the specific modulation of complex I activity by mitochondrial MEF2D. (A) Requirement of mitochondrial MEF2D for complex I activity. Over-expression of Mt2Ddn reduced mitochondrial complex I activity revealed by BN-PAGE and in-gel activity staining (upper panel). Coomassie blue staining showed specific reduction of complex I protein level after over-expression of Mt2Ddn (lower panel) (n=3; **P<0.01). Control indicates the control vector group. (B) Over-expression of ND6 rescued complex I activity reduced by Mt2Ddn. Control indicates the control vector group. (C) Measurement of various complex activities. Quantitative analysis of mitochondrial complex activities showed specific reduction of complex I activity by Mt2Ddn in SN4741 cells (n=4; **P<0.01). Control indicates the control vector group. (D) Effects of over-expression of Mt2D or Mt2Ddn on mitochondrial function. Mt2Ddn reduced cellular ATP level and elevated H₂O₂ production in SN4741 cells (n=4; *p<0.05; **P<0.01). Control indicates the control vector group.

FIG. 6 shows data on inhibition of mitochondrial MEF2D by toxic signals relevant to PD. (A) Reduced binding of MEF2D to ND6 after neurotoxin treatment. SN4741 cells were treated with MPP+ (25 μM) or rotenone (Rot, 100 nM) for 12 hours. ChIP assay showed that binding of MEF2D to ND6 was greatly reduced (n=4; **P<0.01). Control indicates untreated. (B) Reduced mitochondrial MEF2D and ND6 protein levels after neurotoxin treatment. Western blotting showed that levels of MEF2D and ND6 in purified mitochondria (Mt, upper left panel) but not in nuclei (Nu, upper right panel) were significantly reduced (n=4; **P<0.01). Control indicates untreated. (C) Immunocytochemical analysis of mitochondrial MEF2D after MPP+ and rotenone treatment. SN4741 cells treated as described in (A) were stained (scale bar, 10 μm). MPP+ and Rot preferentially reduced the colocalization of MEF2D with MitoTracker (n=50 cells; **P<0.01). Experiments were repeated four times. Control indicates untreated. (D) Effect of mitochondrial MEF2D-ND6 pathway on MPP+ toxicity in SN4741 cells. SN4741 cells were treated with MPP+ after infection with the control, Mt2Ddn or Mt2DVP16 or MtND6 lentiviruses for 24 hours (upper graph: different doses of MPP+ for 24 hours; lower graph: 5 μM of MPP+ for different times). Cell viability was measured by WST-1 assay (n=4; *p<0.05; **P<0.01). Control indicates the control vector group.

FIG. 7 shows data on the correlation of mitochondrial MEF2D in MPTP model of PD and in postmortem brains of PD patients. (A) Reduced mitochondrial MEF2D and ND6 levels in the brains of MPTP treated mice (n=18; **P<0.01). For each group, six eight-week-old mice received chronic intraperitoneal injections of MPTP-HCl in saline or saline alone were killed on 5 or 20 days after injection. Mitochondria purified from brain SNpc region were analyzed by western blotting. Experiments were repeated three times. (B) Role of mitochondrial MEF2D-ND6 pathway in maintaining TH+ neurons in SNpc in a MPTP mouse model of PD. For each group, three mice received stereotactical injection of control vector (GFP) or Mt2Ddn lentivirus in SN. Two weeks later, mice were exposed to MPTP. After treatment for seven days, the survival of lentivirus-transduced TH+ neurons in SN was determined by immunohistochemistry (left panel). Quantitative analysis of nine mice from three independent experiments was shown at the bottom panel (scale bar, 30 μm; **P<0.01). (C) Reduced mitochondrial MEF2D and ND6 levels in the brains of human PD patients. Mitochondria were purified from brain striata of postmortem PD patients and controls (normal). Equal amounts of mitochondrial proteins were subjected to western blotting (upper panel). Quantitative analysis of the bands was shown at the right panel (n=13 patients and 13 controls, *P<0.01). Experiments were repeated two times.

FIG. 8 illustrates FLAG-tagged wild type MEF2D and ΔN30MEF2D. Constructs of FLAG-tagged wild type mouse MEF2D and mutant MEF2D with its N-terminal 30 amino acid (aa) residues deleted (ΔN30MEF2D).

FIG. 9 shows data for targeting Mt2DVP16 and MtND6 to Mitochondria. (A) Diagrams of Mt2DVP16 and MtND6. Mt2DVP16 was constructed by fusion of transactivation domain (TAD) of VP16 with DNA-binding domain of MEF2D and a mitochondria-targeting sequence (Mito). (B) Immunocytochemical staining shows that Mt2DVP16 and MtND6 are targeted exclusively to mitochondria in SN4741 cells (n=3; scale bar, 2.5 μm). (C) Over-expression of Mt2DVP16 attenuated MEF2D siRNA-induced reduction of ND6 expression (n=3, **P<0.01).

FIG. 10 shows data on targeting Mt2D and Mt2Ddn to Mitochondria. (A) Immunocytochemical staining showed that Mt2D and Mt2Ddn were targeted exclusively to mitochondria in SN4741 cells (n=3; scale bar, 2.5 μm). (B) MEF2 reporter assay showed that Mt2D and Mt2Ddn did not affect nuclear MEF2 activity. MEF2D1-131 is a dominant-negative form of MEF2D which interferes with nuclear MEF2 function (n=3, **P<0.01). Control indicates the group co-tranfected with control vector.

FIG. 11 shows data on sensitization of SN4741 cells to MPP+ toxicity by over-expression of Mt2Ddn. (A) SN4741 cells were infected with lentiviruses for 24 hours and followed by exposure to MPP+ (5 μM) for 24 hours. Cell death was analyzed by PI (propidium iodide) staining (n=4; scale bar, 30 μm). The control indicated the control vector. (B) Quantification of the number of PI positive cells in (A) (n=50 cells, **P<0.01). Experiments were repeated four times. Control indicates the group transfected with the control vector.

FIG. 12 shows immunoblots of samples from Parkinson's Disease (PD) patients compared to control patients. (A) samples of whole cell MEF2D levels in lymphoblasts immortalized from PD patients and normal controls were cultured in RPMI-1640 medium with 20% FBS and whole cell proteins were immunoblotted for MEF2D. The membrane was re-probed with and anti-actin antibody for loading control. (B) MEF2D protein levels were measured in mitochondria purified from the immortalized lymphoblast lines and immunoblotted for mitochondrial MEF2D. The membrane was re-probed with an anti-Tom 40 antibody for mitochondrial loading control.

### DETAILED DISCUSSION

It has been discovered that MEF2 plays a role in mitochondria DNA expression. Mitochondrial DNA encoded ND6 gene was identified as the direct target regulated by MEF2D. Immunocytochemical, immunoelectron microscopic, and biochemical analyses show that a portion of MEF2D is targeted to mitochondria via an N-terminal motif and chaperone protein mtHsp70. MEF2D binds to a MEF2 consensus site present in the coding region of the mitochondrial DNA (mtDNA) encoded gene for NADH dehydrogenase 6 (ND6) to regulate its transcription. Blocking MEF2D function specifically in mitochondria decreases complex I activity, increases cellular hydrogen peroxide level, reduces ATP production, and sensitizes neurons to stress-induced death. Toxins known to affect complex I preferentially disrupt MEF2D function in animal model of Parkinson's disease (PD). Consistently, mitochondrial MEF2D and ND6 levels are decreased in brains of PD patients. Thus, direct regulation of complex I by mitochondrial MEF2D underlies its neuroprotective effects. Dysregulation of this pathway may contribute to PD. Disruption of this pathway underlies neurotoxicity induced by toxic signals relevant to PD in culture and animal models, and is found in the brains of PD patients.

### Terms

Unless the context provided otherwise, the term “MEF2” or “MEF2 isoform” refers to the transcriptional regulators MEF2A, MEF2B, MEF2C, or MEF2D, mutants, analogs, or variants, thereof. Generally, the term is not intended to be limited to any particular sequence as long as there is sufficient homology and correlative functional attributes, e.g., MADS box and MEF2 domaine: amino acids approximately 2-86 of SEQ ID NO:1, Holliday junction regulator protein family C-terminal repeat: amino acids approximately 95-155 of SEQ ID NO:1, DNA binding, exist as a dimer, etc. As provided for in GeneBank Accession number NP_{—}005911.1, the amino acid sequence of human MEF2D is SEQ ID NO:1 (MGRKKIQIQR ITDERNRQVT FTKRKFGLMK KAYELSVLCD CEIALIIFNH SNKLFQYAST 61 DMDKVLLKYT EYNEPHESRT NADIIETLRK KGFNGCDSPE PDGEDSLEQS PLLEDKYRRA 121 SEELDGLFRR YGSTVPAPNF AMPVTVPVSN QSSLQFSNPS GSLVTPSLVT SSLTDPRLLS 181 PQQPALQRNS VSPGLPQRPA SAGAMLGGDL NSANGACPSP VGNGYVSARA SPGLLPVANG 241 NSLNKVIPAK SPPPPTHSTQ LGAPSRKPDL RVITSQAGKG LMHHLTEDHL DLNNAQRLGV 301 SQSTHSLTTP VVSVATPSLL SQGLPFSSMP TAYNTDYQLT SAELSSLPAF SSPGGLSLGN 361 VTAWQQPQQP QQPQQPQPPQ QQPPQPQQPQ PQQPQQPQQP PQQQSHLVPV SLSNLIPGSP 421 LPHVGAALTV TTHPHISIKS EPVSPSRERS PAPPPPAVFP AARPEPGDGL SSPAGGSYET 481 GDRDDGRGDF GPTLGLLRPA PEPEAEGSAV KRMRLDTWTL K). The 30 N-terminal amino acids are SEQ ID NO:2 (MGRKKIQIQR ITDERNRQVT FTKRKFGLMK).

As provided for in GeneBank Accession number NP_{—}598426.1, the amino acid sequence of mouse MEF2D is SEQ ID NO:3 (MGRKKIQIQR ITDERNRQVT FTKRKFGLMK KAYELSVLCD CEIALIIFNH SNKLFQYAST 61 DMDKVLLKYT EYNEPHESRT NADIIETLRK KGFNGCDSPE PDGEDSLEQS PLLEDKYRRA 121 SEELDGLFRR YGSSVPAPNF AMPVTVPVSN QSSMQFSNPS SSLVTPSLVT SSLTDPRLLS 181 PQQPALQRNS VSPGLPQRPA SAGAMLGGDL NSANGACPSP VGNGYVSARA SPGLLPVANG 241 NSLNKVIPAK SPPPPTHNTQ LGAPSRKPDL RVITSQGGKG LMHHLNNAQR LGVSQSTHSL 301 TTPVVSVATP SLLSQGLPFS SMPTAYNTDY QLPSAELSSL PAFSSPAGLA LGNVTAWQQP 361 QPPQQPQPPQ PPQSQPQPPQ PQPQQPPQQQ PHLVPVSLSN LIPGSPLPHV GAALTVTTHP 421 HISIKSEPVS PSRERSPAPP PPAVFPAARP EPGEGLSSPA GGSYETGDRD DGRGDFGPTL 481 GLLRPAPEPE AEGSAVKRMR LDTWTLK).

### Method of Diagnosis and kit

The disclosure relates to methods of identifying a subject who has or is at risk of developing a disease related to mitochondrial dysfunction, and in particular, Parkinson's disease (PD), and other related diseases. In certain embodiments, a method of diagnosing a disease related to mitochondrial dysfunction in a subject is provided including the steps of analyzing a sample for at least one mitochondrial myocyte enhancer factor 2 (MEF2) isoform level, wherein low levels of the MEF2 isoform indicate a disease in the subject.

In certain embodiments, the sample is obtained from a subject and exposed to at least one reagent that indicates presence of a MEF2 isoform. In further embodiments, a kit is provided including reagents that provide an indication of MEF2 isoform levels. In certain instances, the reagents will provide an output when levels of an MEF2 isoform are below a threshold value. In certain embodiments, reagents may be included to detect levels of at least two MEF2 isoforms. In certain other embodiments, reagents may detect at least three or more MEF2 isoforms. In specific embodiments, the reagents can be quantified for levels of MEF2 isoforms in a mitochondria.

In certain embodiments, level of a MEF2 isoform is compared to a control value. In certain embodiments, MEF2 isoform levels in the sample are reduced at least by 40% or over compared with corresponding control levels. In certain embodiments, MEF2 isoform levels are reduced by at least 45% or at least 50% or at least 55% or at least 60% or at least 65% or at least 70% over control levels. In certain embodiments, level of ND6 is compared to a control value. ND6 levels in the sample are reduced at least by 40% over compared with corresponding control levels. In certain embodiments, ND6 isoform levels are reduced by at least 45% or at least 50% or at least 55% or at least 60% or at least 65% or at least 70% over control levels.

In certain embodiments, the control level is the amount of a MEF2 isoform in a sample of a non-disease subject. In some embodiments, the sample is from blood. In some embodiments, the sample is mitochondria of a non-disease subject or a value obtained from a population of non-disease subjects, such as at least 20, at least 40 or at least 100 non-disease subjects. In certain embodiments, the control level is a level of whole-cell MEF2 isoform or ND6 and the sample is a sample of mitochondrial MEF2 isoform or ND6. In certain embodiments, the method further includes a step of isolating mitochondria from the sample. In certain embodiments, a sample is taken from a subject and a portion of the sample is reacted with a reagent detecting a MEF2 isoform and another portion of the sample is subjected to isolation of mitochondria, which are then reacted with the same reagent.

In certain embodiments, protein levels are measured. In other embodiments, mRNA levels are measured. In certain embodiments, levels are measured as either protein or mRNA per microgram of a standard, such as actin.

In some embodiments, the MEF2 isoform is MEF2D. In certain embodiments, the MEF2 isoforms are measured using an antibody reactive with an isoform, such as MEF2D. Such an antibody can be labeled with an agent for measurement, such as luciferase, or it can be labeled with another reactive moiety that allows measurement through further reaction, such as biotin/avidin reactions. In other embodiments, the reagent is a non-antibody protein that binds to a MEF2 isoform. In certain embodiments, the reagent allows visualization if the level of the MEF2 isoform is below a threshold value, or if it is below the level in a control such as a whole cell sample. In other embodiments, the reagent allows visualization if the level is at or above such a control. In certain embodiments, the mitochondria are isolated from the sample prior to analysis. In certain embodiments, the kit includes a further reagent to identify mitochondria.

In certain embodiments, the disclosure relates to methods of diagnosing Parkinson's disease or related disease in a subject comprising analyzing a sample for mitochondrial MEF2 isoform levels and correlating low levels of MEF2 isoform to Parkinson's disease or a related disease in the subject. Typically the MEF2 isoform is MEF2A, MEF2B, MEF2C, and/or MEF2D. In certain embodiments, the method further comprises the step of analyzing the sample for cytoplasmic MEF2 and correlating high levels of MEF2 to Parkinson's disease or a related disease in the subject. In certain embodiments, the disclosure relates to methods of diagnosing Parkinson's disease or related disease in a subject comprising analyzing a sample for mitochondrial MEF2D and correlating low levels of MEF2D to Parkinson's disease or a related disease in the subject. (Note: it depends individual case, wherein we decide to delete the following description).

Generally, the MEF2 isoforms are mitochondrial MEF2A, MEF2B, MEF2C, and/or MEF2D. The isoform levels can be analyzed in a sample that is in peripheral blood, wherein the sample includes peripheral blood cells and white blood cells. In other embodiments, the sample is a neuronal sample and mitochondrial MEF2A, MEF2B, MEF2C, and/or MEF2D levels are measured in this sample and correlated the levels to mitochondrial impairment, disease diagnosis, treatment, and/or prognosis.

In typical embodiments, the disease is Alzheimer's disease, Parkinson's disease, Huntington's disease, amyotrophic lateral sclerosis, hereditary spastic paraplegia, cerebellar degenerations, diabetes mellitus, deafness, Leber's hereditary optic neuropathy, neuropathy, ataxia, retinitis pigmentosa, ptosis, myoneurogenic gastrointestinal encephalopathy, or myoclonic epilepsy with ragged red fibers, or the subject has mitochondrial myopathy, encephalomyopathy, lactic acidosis, or stroke-like symptoms.

In certain embodiments, the disclosure relates to analyzing a sample for MEF2 isoform levels such as, MEF2A, MEF2B, MEF2C, and/or MEF2D, and correlating aberrant expression to the presence of related diseases.

### Methods of Treatment

In certain embodiments, the disclosure relates to directly enhancing mitochondrial MEF2A, MEF2B, MEF2C, and/or MEF2D levels as a method of treating related diseases. In certain embodiments, the disclosure relates to targeted delivery of MEF2 isoforms specifically to mitochondria to enhance mitochondrial function in therapy. In specific embodiments, the MEF2 isoform is a MEF2D isoform. In specific embodiments, the level of a native MEF2 isoform is enhanced in a cell. In other embodiments, a level of a MEF2 isoform is enhanced by external administration.

MEF2 mutants have been developed that can be targeted specifically to mitochondria instead of the cell nucleus. Depending on the form of MEF2 (active or dominant negative), it can either block or enhance endogenous mitochondrial MEF2 function. MitoMEF2 (Mt2Dwt or Mt2Ddn) can be used therapeutically to modulate mitochondrial function in cells. In certain embodiments, the disclosure relates to pharmaceutical compositions comprising MEF2 isoforms and mutant forms and methods of treating or preventing related diseases by administering the pharmaceutical composition comprising a MEF2 isoform to a subject at risk of, exhibiting symptoms of, or diagnosed with the disease.

In certain embodiment, the disclosure relates to isolated chimeric proteins comprising 1) a MEF2 isoform such as MEF2A, MEF2B, MEF2C, and/or MEF2D sequence and 2) a mitochondrial-targeting signal. Typically, the MEF2 sequence lacks a nuclear localization signaling sequence and the second mitochondrial-targeting signal is conjugated to the N-terminus of the MEF2 sequence. In certain embodiments, the MEF2 sequence comprises a mutation. The 30 N-terminal amino acids of wild-type MEF2D contain a mitochondrial targeting signal. This is the endogenous signal of wild-type MEF2D. It is contemplated that a chimeric protein contains an extra targeting signal at the N-terminus, i.e. the endogenous targeting signal and a second targeting signal. In certain embodiments, the disclosure relates to isolated chimeric proteins comprising SEQ ID NO: 2 wherein said isolated chimeric protein does not contain a TAD, MADS box and MEF2 domain, and/or Holliday junction regulator protein family C-terminal repeat. In certain embodiments, the disclosure relates to nucleic acids that encode chimeric proteins disclosed herein. In certain embodiments, the disclosure relates to recombinant vectors such adenoviruses, adeno-associated viruses, plasmids, and lentiviruses that encode nucleic acids that transcribe chimeric proteins disclosed herein.

In some embodiments, the disclosure relates to an isolated chimeric protein comprising, 1) a MEF2D sequence and 2) a second mitochondrial-targeting signal wherein the MEF2D sequence lacks a nuclear localization signaling sequence. In a typically embodiment, the second mitochondrial-targeting signal is conjugated to the N-terminus of the MEF2D sequence which is the 30 N-terminal amino acid residues of MEF2D. In certain embodiments, the MEF2D sequence comprises mutation. In certain embodiments, the disclosure relates to methods of treating Parkinson's Disease or related disease comprising administering a pharmaceutical composition comprising a protein comprising a MEF2D sequence to a subject in need thereof. Typically, the protein is a chimeric protein comprising, 1) a ME2D sequence and 2) a second mitochondrial-targeting signal, wherein said protein is lacking a nuclear localization signaling sequence.

In certain embodiments, the disclosure relates to methods of diagnosing Parkinson's disease or related disease in a subject comprising analyzing a sample for MEF2 mitochondrial target gene ND6 levels and correlating low levels of ND6 to Parkinson's disease or a related disease in the subject by using RT-PCR with ND6 specific primers (forward, 5′-ATTAAACAA CCAACAAACCCAC-3′, reverse, 5′-TTTGGTTGGTTGTCTTGGGTT-3′) or western blotting with anti-ND6 antibody. In certain embodiments, the disclosure relates to methods of treating diseases related to mitochondrial dysfunction by enhancing ND6 function or expression. In certain embodiments, the molecule is ND6 gene or ND6 protein. In a typical embodiment, the gene or protein is in or expressed in a recombinant vector. In certain embodiments, the disclosure relates to isolated chimeric proteins comprising ND6 and a mitochondrial-targeting signal or nucleic acids that encode such proteins. In certain embodiments, the disclosure relates to methods of treating diseases related to mitochondrial dysfunction comprising administering a pharmaceutical composition comprising a molecule that inhibits or enhances ND6 or ND6 gene expression. In certain embodiments, the molecule is a small molecule agonist or antagonist of ND6, ND6 antibody, aptamer, or siRNA of the ND6 gene.

### MEF2 and MEF2D in Mitochondrial DNA Expression

Nuclear transcription factor MEF2s are involved in a growing number of critical cellular functions involving both neuronal and non-neuronal systems. The basic assumption has always been that MEF2s exert their control on cells solely through modulating the expression of nuclear target genes. Indeed, MEF2A has been shown to affect mitochondrial function by regulating the expression of mitochondrial proteins encoded by the nuclear genes. This disclosure provides the first evidence to show that MEF2D is present in neuronal mitochondria, where it binds to a discrete and well conserved MEF2 consensus site within the coding region of mitochondrial gene ND6 to regulate its transcription, thereby directly modulating complex I activity and affecting a number of key mitochondrial functions and physiology. Thus, MEF2D qualifies as a bona fide, mitochondrial transcription factor. These findings broaden the cellular roles played by MEF2D.

Yang et al., Science, 2009, 323:124-127 disclose that the autophagic pathway regulates the activity of nuclear MEF2D. Whether autophagy plays a similar role in controlling mitochondrial MEF2D activity was examined. While MEF2D levels in the whole cell are elevated in the brains of alpha-synuclein transgenic mice and PD patients, its levels in the mitochondria are decreased in PD patients. Collectively, these data indicate that the decrease in MEF2D level in mitochondria is accompanied by a buildup of MEF2D in the cytoplasm. It is possible that reduced mitochondrial MEF2D may contribute to the overall increase of cytoplasmic MEF2D. But this effect should be small since only a small fraction of MEF2D will normally go to mitochondria.

Data disclosed herein suggests that MEF2D exclusively regulates the expression of the ND6 gene without significant effects on several other protein-encoding mtDNA genes tested. Since ND6 is the only protein-encoding gene present in the L strand of mtDNA while the rest of the thirteen protein-encoding mitochondrial genes all reside in the H strand, this apparent specificity may be in part due to the unique organization of mtDNA. Indeed, cAMP response element binding (CREB) protein has been shown to bind the D-loop, while p53 can also localize to mitochondria under stressful conditions. But none of them has been reported to affect L strand transcription. Insufficiency of ND6 protein is known to lead to severe disruption of complex I structure. Therefore, maintaining adequate levels of ND6 is important for the proper assembly of complex I. Data disclosed herein shows that a reduction of MEF2D activity specifically in mitochondria will result in significant disorganization of complex I and subsequent significant loss of complex I activity without affecting other complexes. These findings highlight the distinctive role of MEF2D in maintaining the function of complex I. Together with the reported mitochondrial localization by CREB and p53, these studies emphasize a previously underappreciated mechanism of interaction between nucleus and mitochondria. The selective degeneration of DA midbrain neurons in the substantia nigra (SN) is a hallmark of Parkinson disease. DA neurons in the neighboring ventral tegmental area (VTA) are significantly less affected. The mechanisms for this differential vulnerability of DA neurons are unknown. Recent studies have identified several differences between them, including different transcriptional response to MPTP, divergent electrophysiological features, and selective activation of ATP-sensitive potassium (K-ATP) channels. It is, therefore, thought that SN and VTA DA neurons may have different sensitivity to mitochondrial malfunction induced by MEF2D defects.

Studies disclosed herein offer multiple lines of evidence including cellular model, animal studies, and human tissues to implicate a regulatory mechanism in the pathogenic process of Parkinson's disease. Well-established toxins known to target complex I and induce parkinsonism in model systems also reduce MEF2D levels in mitochondria and disrupt MEF2D binding to the ND6 MEF2 site, thus offering an alternative mode of action by which these important toxins inhibit mitochondrial function. Since low dose toxins preferentially reduce mitochondrial MEF2D without affecting its level in the nucleus, data disclosed herein suggests that inhibition of the MEF2D-ND6 pathway represent one of the earlier steps involved in pathologic changes at subcellular level. More importantly, the levels of both MEF2D and ND6 proteins are greatly reduced in brain mitochondria of both chronic MPTP-treated mice and human PD patients. These findings are consistent with the notion that reduced complex I activity secondary to toxic agent-induced inhibition of the MEF2D-ND6 pathway may contribute to the mitochondrial dysfunction and oxidative stress often observed in PD and possibly in other neurodegenerative diseases. Since both MPP+ and rotenone directly bind complex I, it is thought that these toxins may exert their effects on MEF2D via a mechanism involving complex I inhibition or disruption of mitochondrial redox balance.

Kim et al., disclosed that mice with MEF2D gene conditionally deleted are viable and show no obvious phenotypic abnormalities. J Clin Invest, 2008, 118:124-132. However, under stress, these mice showed defects in cardiac remodeling. This is consistent with data disclosed herein demonstrating that reduced MEF2D activity in mitochondria sensitizes the cells to toxic stress. It is contemplated that mitochondrial MEF2D plays a role in other organ systems and disease processes as well.

### Localization of MEF2D in Mitochondria of Neuronal Cells

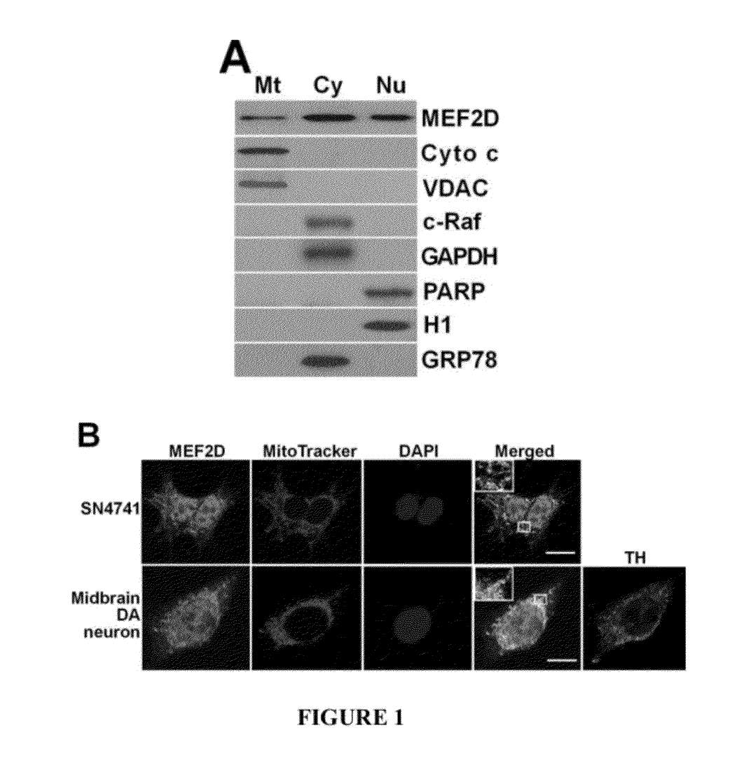

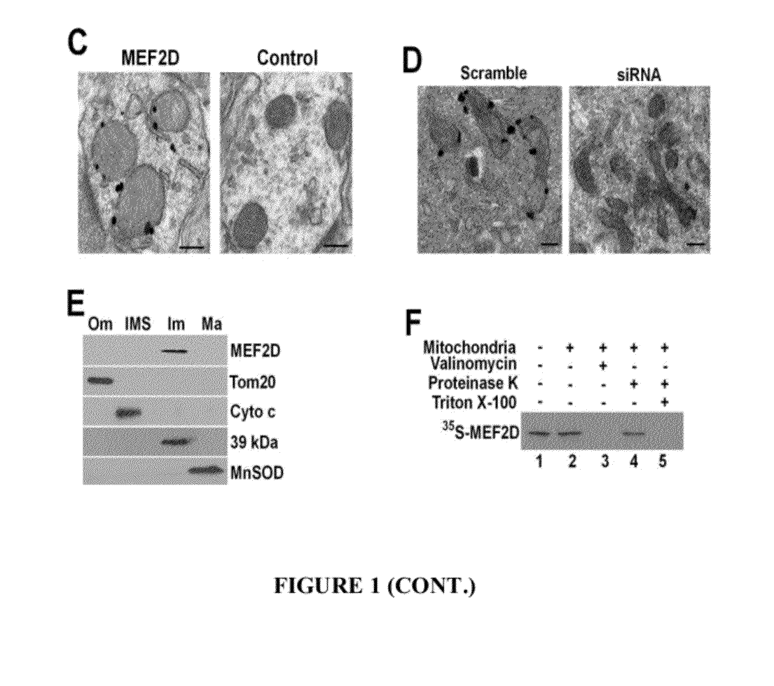

To determine mitochondrial localization of MEF2D, purified mitochondrial fractions were prepared from SN4741 cells, a mouse dopaminergic (DA) neuronal cell line expressing tyrosine hydroxylase (TH) and widely used in the study of neuronal toxins. See Son et al., J Neurosci, 1999, 19:10-20. Western blot analysis showed that MEF2D is present in highly purified mitochondrial fractions (FIG. 1A). The cytoplasmic (c-Raf and GAPDH) and nuclear (PARP and H1) markers were only detected in their respective fractions, indicating that the mitochondrial preparations were not contaminated with other subcellular fractions. The monoclonal antibody used to probe MEF2D was very specific as it recognized a single band on western blot, which was reduced to background by MEF2D siRNA. Consistent with western blot findings, immunocytochemical studies confirmed the colocalization of MEF2D with a mitochondrial specific fluorescent dye MitoTracker in cultured SN4741 cells and primary midbrain dopamine neurons (FIG. 1B). MEF2D siRNA reduced MEF2D-MitoTracker colocalization signal.

The ultrastructural distribution of MEF2D by immunogold electron microscopy (EM) was investigated. Specific immunogold particles were preferentially found in mitochondria of rat brain and SN4741 cells. This immunogold particle distribution was not observed when the primary anti-MEF2D antibody was omitted and was greatly reduced after MEF2D siRNA (FIGS. 1, C and D). To localize MEF2D within mitochondria biochemically, subfractionation of highly purified rat brain mitochondria was performed. Western blot analysis of the subfractions showed that MEF2D is present predominately in the inner membrane of mitochondria (FIG. 1E). In vitro mitochondrial import assay showed that MEF2D translated in vitro is imported into isolated energized mitochondria (FIG. 1F). Collectively, these data demonstrate that a portion of MEF2D in neurons is localized to mitochondria, and enriched in the mitochondrial inner membrane.

### Specific Sequence and Chaperone Protein Required for Localization of MEF2D to Mitochondria

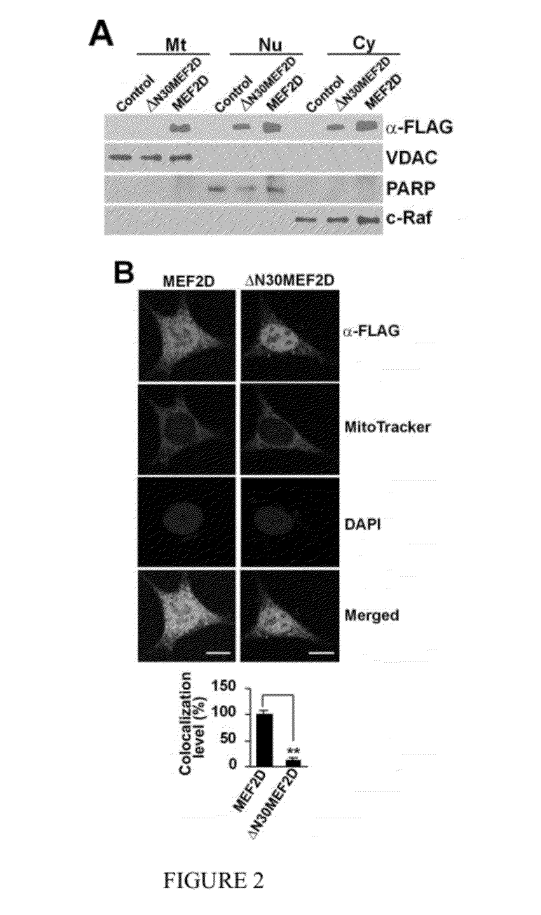

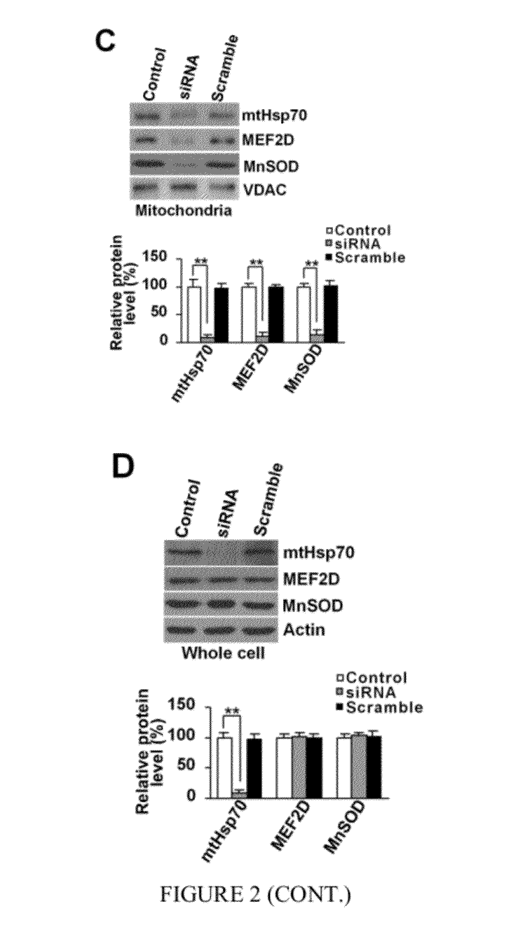

Proteins are often targeted to mitochondria if their sequence contains a mitochondrial-targeting motif, and sometimes requires the aid of chaperones. The structure of MEF2D is divided into the smaller N-terminal DNA binding domain and the larger C-terminal transactivation domain (TAD, amino acids approximately 87-521 of SEQ ID NO:1). The N-terminal 30 amino acid (aa) residues of MEF2D are involved in protein interactions including chaperones and homology to a motif for mitochondrial localization signal by iPSORT (http://hc.ims.u-tokyo.ac.jp/iPSORT/). A MEF2D-Flag mutant was generated with the putative mitochondrial targeting signal deleted (ΔN30MEF2D) and studied its subcellular distribution. Wild type MEF2D-Flag, when over-expressed in SN4741 cells, was detectable in nuclear, cytoplasmic and mitochondrial fractions just as the endogenous MEF2D (FIG. 2A and FIG. 1A). When transfected into cells, the ΔN30MEF2D-Flag mutant was well expressed and readily detectable in whole cell lysate. But unlike its wild type counterpart, ΔN30MEF2D-Flag mutant was absent from the mitochondrial fraction but detectable in the cytoplasmic and nuclear fractions (FIG. 2A). Consistent with this, immunocytochemical studies showed that in contrast to wild type MEF2D-Flag, ΔN30MEF2D-Flag largely lost its co-localization with the mitochondrial marker MitoTracker in SN4741 cells (FIG. 2B).

Mitochondrial heat shock protein (mtHsp70) is a key component of the mitochondrial import machinery. Whether blocking mtHsp70 affects localization of MEF2D to mitochondria was tested by transfecting SN4741 cells with siRNA oligos targeting mouse mtHsp70 and then examining MEF2D in purified mitochondria. Mouse mtHsp70 siRNA led to a marked reduction of mtHsp70 protein level. This was accompanied by a similar decline in the level of mitochondrial MEF2D and MnSOD, the latter known to require mtHsp70 for its mitochondrial localization (FIG. 2C). On the other hand, the level of MEF2D in whole cell lysate was not affected (FIG. 2D), indicating that translocation of MEF2D into mitochondria is specifically regulated by mtHsp70.

### Identification of MEF2D Regulatory Target in Mitochondrial DNA

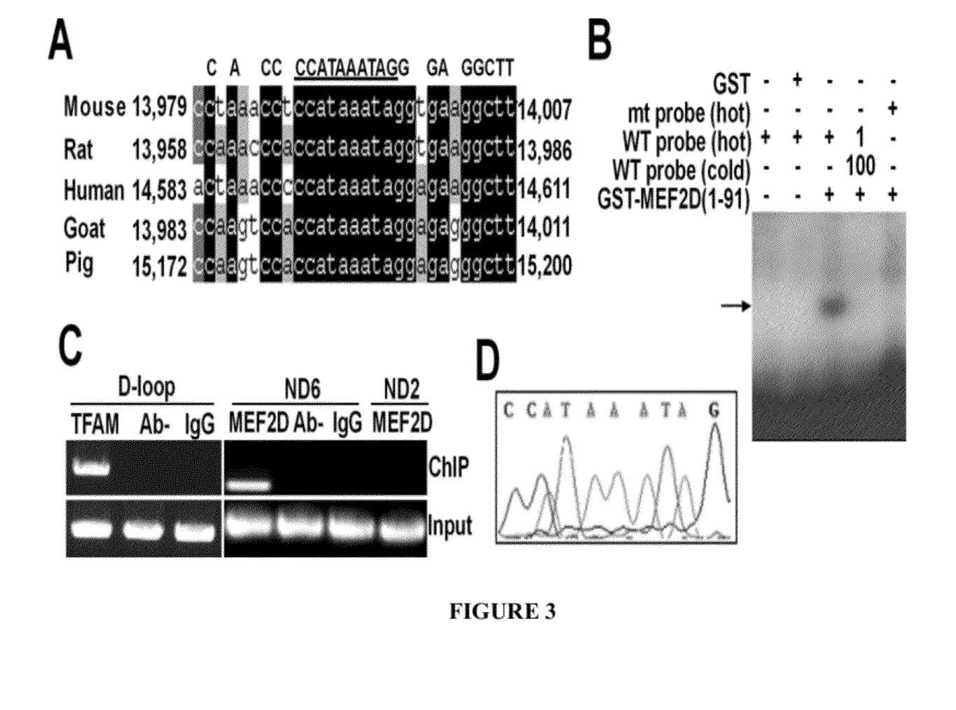

Analyzing the entire mtDNA revealed the presence of a single putative MEF2 consensus site (5′-CC(A/t)(t/a)AAATAG-3′) in the coding region of the ND6 gene (FIG. 3A). Andres et al., J Biol Chem, 1995, 270:23246-23249. Moreover, this putative MEF2 binding site is conserved among several species. To test whether MEF2D binds to this putative MEF2 site, electronic mobility shift assay (EMSA) was performed. N-terminal MEF2D formed a complex with the labeled probe containing the ND6 MEF2 site. Mutation of the ND6 MEF2 site disrupted the complex formation, suggesting that MEF2D specifically binds to this site in vitro (FIG. 3B). To corroborate this finding, a chromatin immunoprecipitation (ChIP) assay was carried out using highly purified mitochondria. ChIP analysis showed that MEF2D binds specifically to this ND6 region in SN4741 cells. ND6 but not ND2 based primers could amplify the bound sequence (FIG. 3C). Sequence analysis of the DNA bound by MEF2D after amplification confirmed the presence of the ND6 MEF2 site (FIG. 3D).

### Transcriptional Regulation of Mitochondrial Gene ND6 by Mitochondrial MEF2D

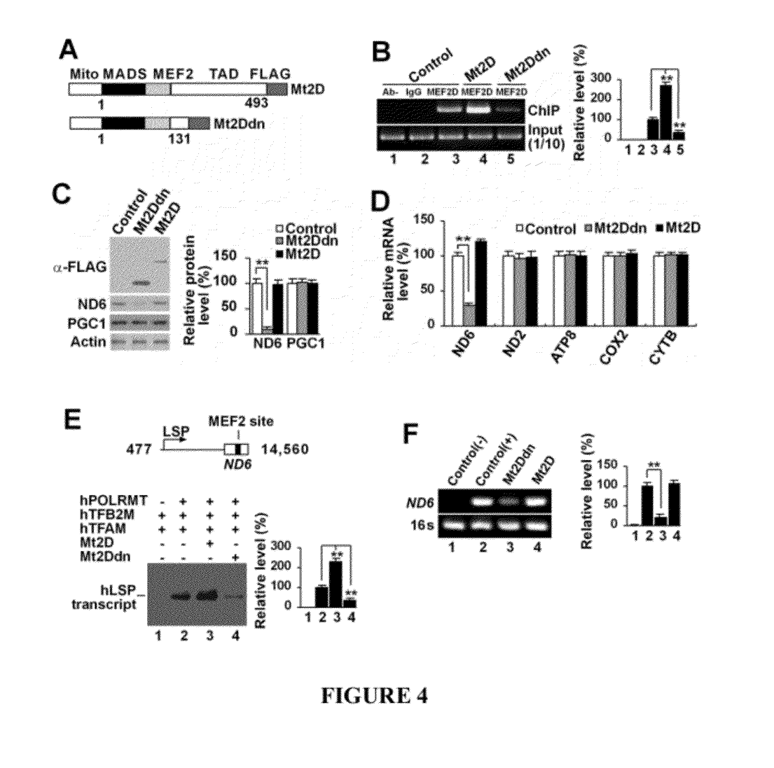

To assess the function of MEF2D specifically in mitochondria without affecting nuclear MEF2, MEF2D mutants were generated (Mt2D, active MEF2D; Mt2Ddn, dominant negative MEF2D) that lack the nuclear localization signals at the very C-terminus of the protein and carry an additional mitochondria-targeting signal fused to the N-terminus (FIG. 4A). These mutants display an enhanced ability to preferentially localize to the mitochondria versus the nucleus. When over-expressed in SN4741 cells, they colocalized with MitoTracker in the cytoplasm with greatly reduced nuclear presence as analyzed by immunocytochemistry. MEF2 reporter assay, which measures nuclear MEF2 activity, showed that unlike their nuclear counterparts, neither the active nor the dominant negative mitochondria-targeted MEF2D mutants affected the expression of nuclear MEF2 reporter. Both Mt2D and Mt2Ddn, when over-expressed in SN4741 cells, modulated the binding of full length MEF2D to the ND6 MEF2 site as shown by ChIP assay (FIG. 4B). Interestingly, although it bound to the ND6 MEF2 site at a high level, Mt2D did not significantly change the level of ND6 protein. In contrast, Mt2Ddn caused a marked reduction of endogenous ND6 protein level (FIG. 4C). Furthermore, Mt2Ddn did not alter the levels of PGC1, a known nuclear target of MEF2, suggesting that Mt2Ddn preferentially changes the MEF2D target in mitochondria but not in the nucleus. ND6 is a key component for oxidative phosphorylation complex I. To assess whether the effect of Mt2Ddn on ND6 is specific, the mRNA levels for the components of various other oxidative phosphorylation complexes were determined. Mt2Ddn had little effect on the mRNA levels of other mitochondria-encoded proteins tested under the condition when it clearly reduced ND6 mRNA (FIG. 4D). To further confirm mitochondrial MEF2D is required for ND6 expression, MEF2D was knocked down by siRNA in SN4741 cells and found that ND6 level is reduced. An active MEF2D targeted to mitochondria and not affected by MEF2D siRNA (Mt2DVP16) completely blocked MEF2D siRNA-induced reduction of ND6 level. To explore how MEF2D regulates ND6 gene expression, in vitro mtDNA L-strand transcription assay was performed. Data showed that Mt2D increases while Mt2Ddn reduces the level of L-strand promoter (LSP) transcripts (FIG. 4E). Consistent with these data, Mt2Ddn reduced the de novo transcription of ND6 in vivo (FIG. 4F). These findings suggest that mitochondrial MEF2D activity is specifically required for the transcription of mitochondrial ND6 gene driven by L-strand promoter.

### Specific Modulation of Complex I Activity by Mitochondrial MEF2D

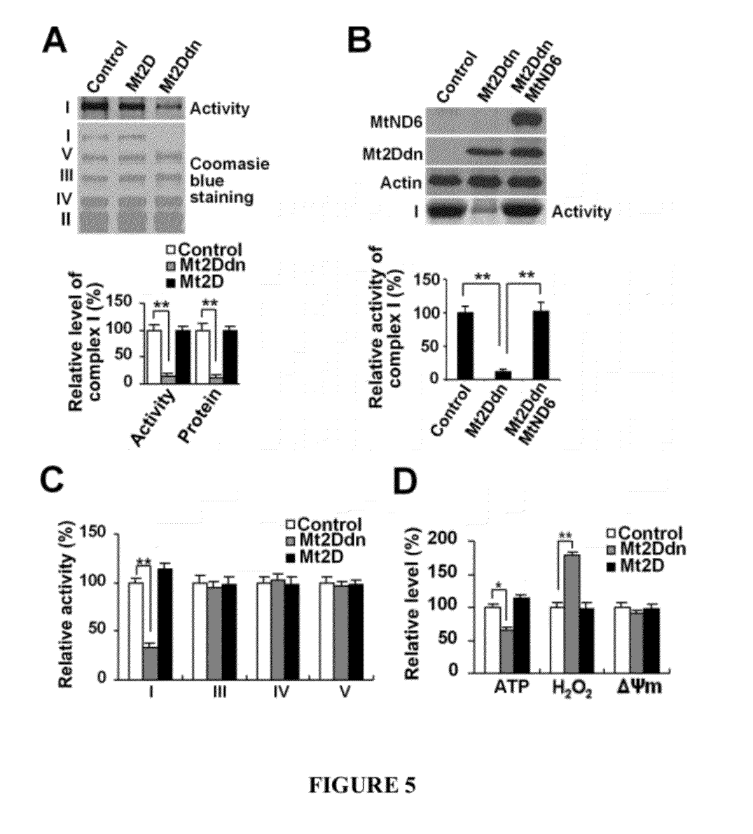

ND6 has been reported to be required for the proper assembly of complex I. Whether activity of mitochondrial MEF2D affects complex I function was tested. Over-expression of Mt2Ddn in SN4741 cells markedly reduced the protein level of mitochondrial complex I but had no effect on complex II-V by non-denature gel electrophoresis (FIG. 5A). Similarly, Mt2Ddn also significantly reduced mitochondrial complex I activity but not complex III-V (FIGS. 5, A and C). Interestingly, Mt2D neither increased the protein level nor the activity of complex I (FIGS. 5, A and C). To further demonstrate that mitochondrial MEF2D-ND6 is required for maintaining complex I activity, ND6 was overexpressed via a construct which was not regulated by mitochondrial MEF2D, and found that this rescues complex I activity from Mt2Ddn-induced inhibition (FIG. 5B). Together, these results demonstrate that the activity of mitochondrial MEF2D is specifically required for complex I function. To corroborate the role of MEF2D in regulation of complex I, whether MEF2D affects mitochondrial functions was determined. Mt2Ddn led to a clear reduction in cellular ATP level and significantly increased hydrogen peroxide production by mitochondria. But it did not significantly alter the mitochondrial membrane potential (FIG. 5C). Consistent with findings in FIGS. 5A and 5B, Mt2D did not alter the levels of ATP, hydrogen peroxide, and membrane potential. These data indicate that direct regulation of ND6 gene by MEF2D is critical and required for proper mitochondrial function.

### Inhibition of Mitochondrial MEF2D by Toxic Signals Relevant to PD

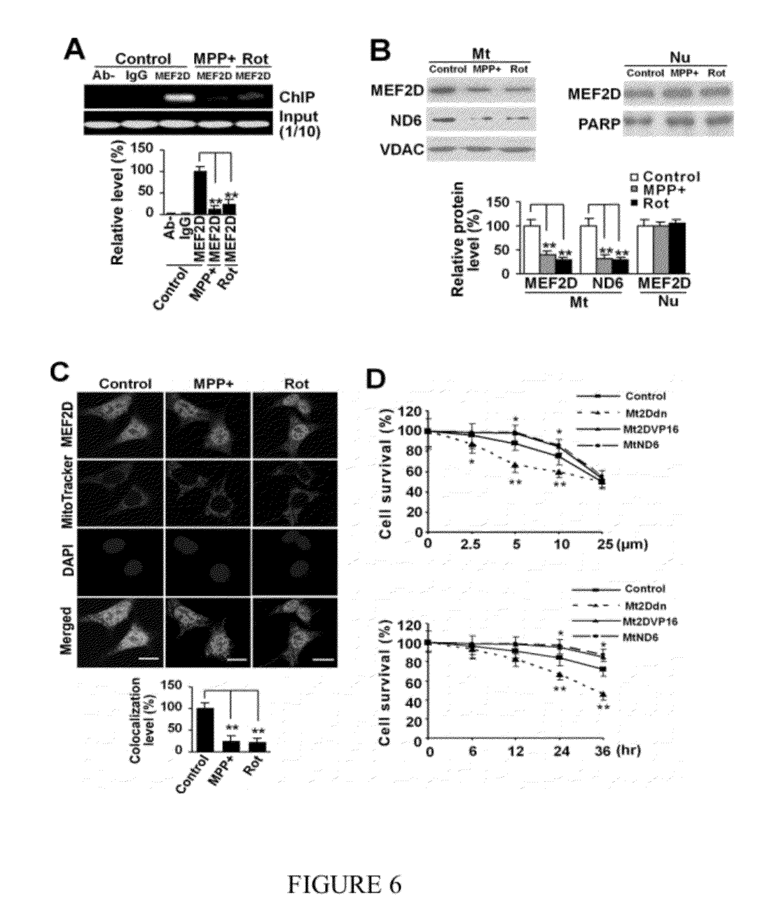

Mitochondrial dysfunction and oxidative stress have been implicated in the pathogenesis of PD. Abou-Sleiman et al., Nat Rev Neurosci, 2006, 7:207-219. This led us to test whether toxic signals implicated in PD regulate mitochondrial MEF2D. SN4741 were treated cells with toxicants 1-methyl-4-phenylpyridinium (MPP+) or rotenone and performed ChIP assays. Relatively short exposure to these toxicants greatly reduced the binding of mitochondrial MEF2D to the ND6 MEF2 site (FIG. 6A) and mRNA level of ND6. Consistently, exposure to MPP+ and rotenone also led to a decrease in the levels of mitochondrial MEF2D and ND6 proteins (FIG. 6B, left panel) while under the same condition the levels of MEF2D in the nucleus (FIG. 6B, right panel) and in whole cell were not affected. To support these western blot findings, immunocytochemistry studies in SN4741 cells and in cultured primary midbrain dopamine neurons were performed. MPP+ and rotenone preferentially reduced the colocalization of MEF2D and MitoTracker signals in these neuronal cells (FIG. 6C).

To assess the role of mitochondrial MEF2D in neuronal survival, SN4741 cells were exposed to different doses of MPP+, known to cause selective degeneration of dopaminergic neurons in vivo, and measured cellular viability by WST-1 assay. MPP+ caused loss of neuronal viability in a dose dependent manner (FIG. 6D). Blocking mitochondrial MEF2D with Mt2Ddn clearly sensitized the cells to low dose MPP+ toxicity as measured by WST-1 assay (FIG. 6D, top panel). Mt2Ddn also impaired cellular viability over time (FIG. 6D, bottom panel). Consistent with these results, immunocytochemical studies revealed that Mt2Ddn increases the number of propidium iodide (PI) positive cells following MPP+ treatment. In agreement with the findings that constitutively active Mt2DVP16 enhances ND6 expression and overexpression of MtND6 restores complex I activity (FIG. 5B), over-expression of Mt2DVP16 or MtND6 in mitochondria partially attenuated MPP+-induced neuronal death (FIG. 6D).

### Correlation of Mitochondrial MEF2D in MPTP Model of PD and in Postmortem Brains of PD Patients

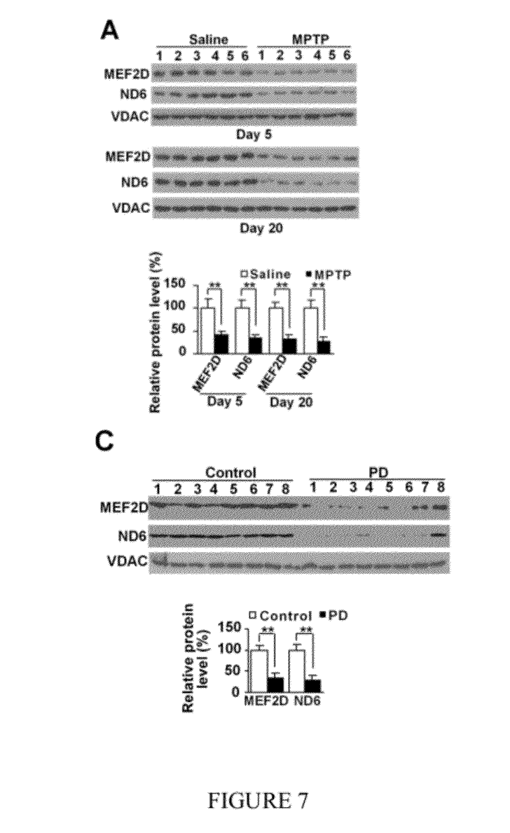

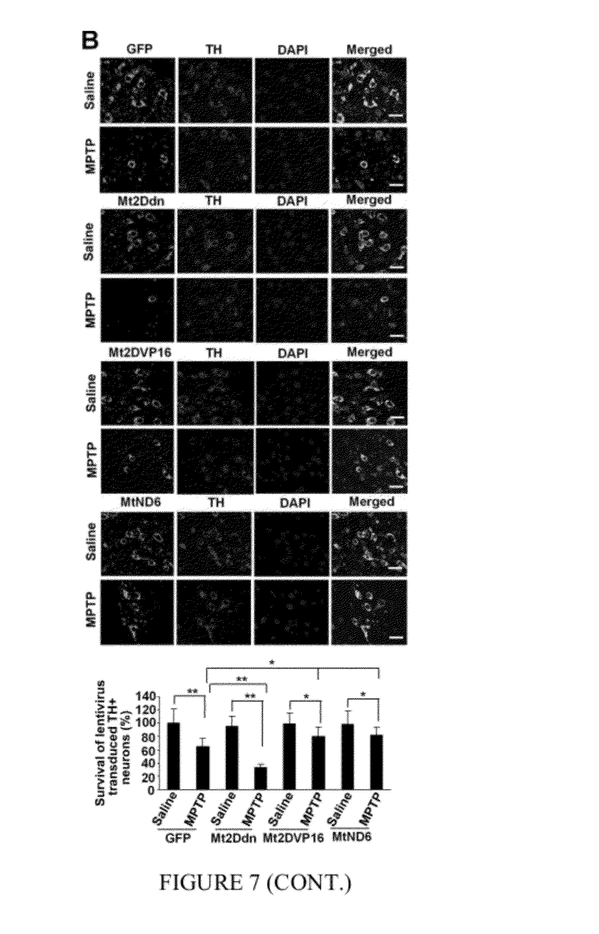

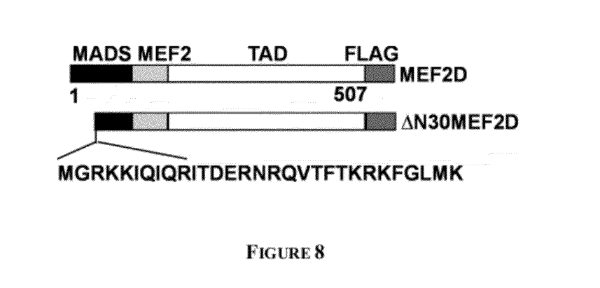

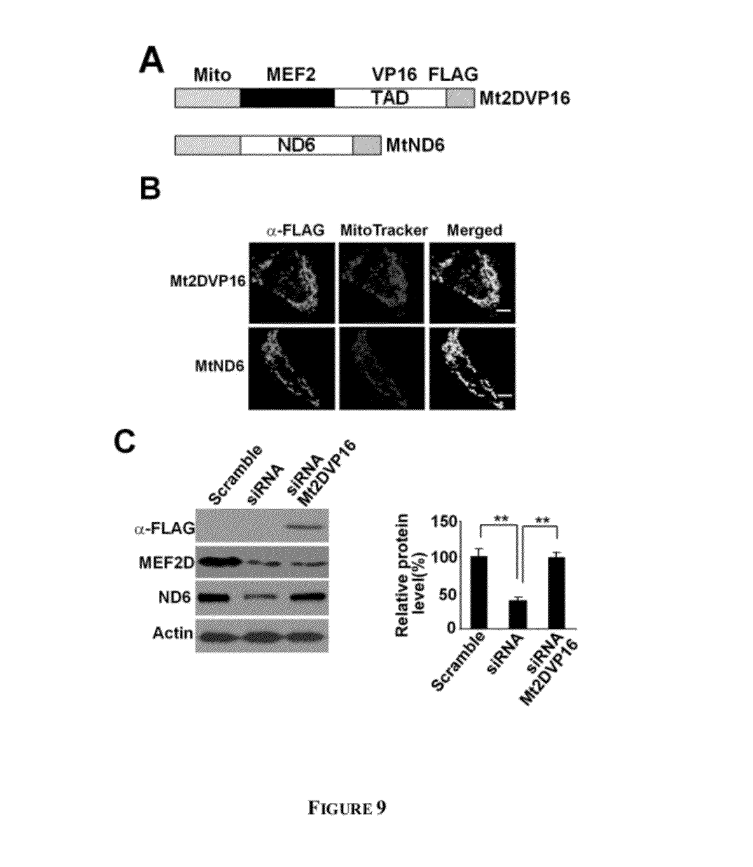

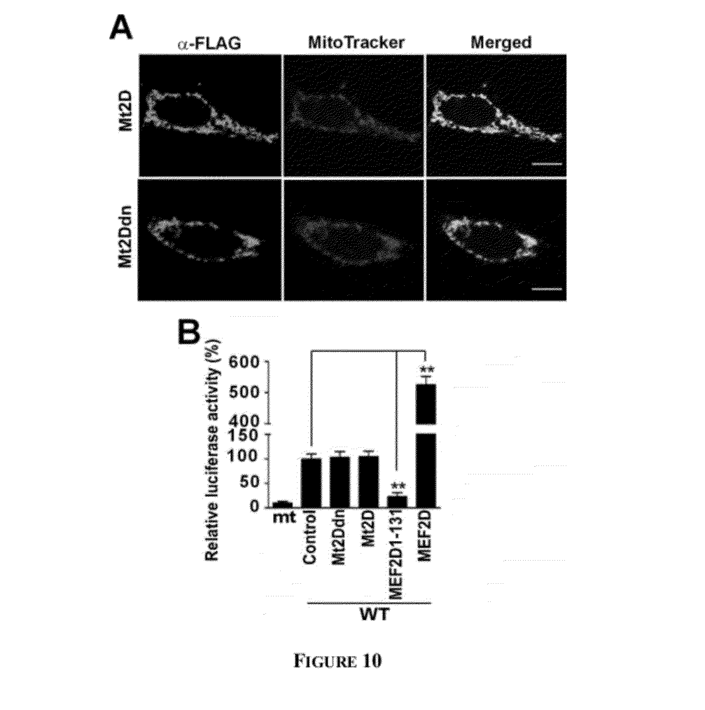

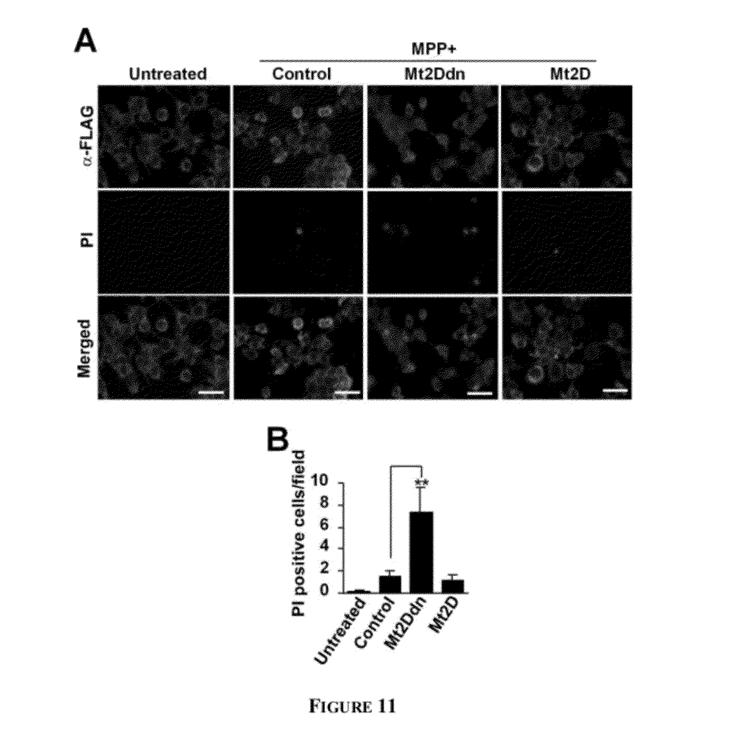

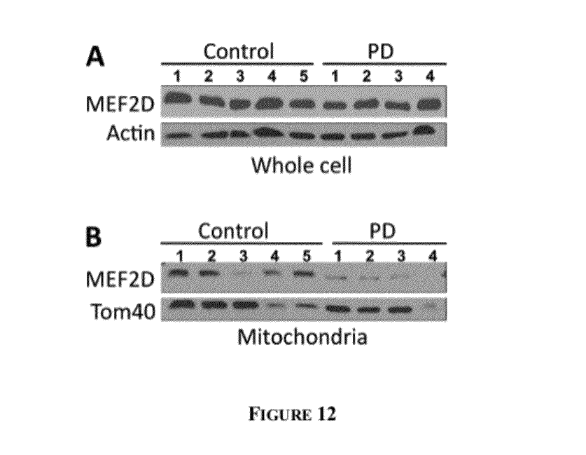

Mice treated with neurotoxin MPTP exhibit classic pathological and behavioral features observed in Parkinsonism and are widely used to model the disease. The effects of this toxin on mitochondrial MEF2D in vivo using the brains of MPTP-treated mice were examined. Chronic MPTP exposure caused a loss of immunohistochemical signal for tyrosine hydroxylase (TH) in SNpc and striatum. This correlated well with a significant reduction of mitochondrial MEF2D in MPTP treated mouse brain compared to saline treated controls. Furthermore, MPTP also led to a clear decline in the level of ND6 protein (FIG. 7A). To show that inhibition of mitochondrial MEF2D plays a direct role in the loss of DA neurons in animal model of PD, recombinant lentivirus encoding Mt2Ddn, Mt2DVP16 or MtND6 was injected into the SNpc of mouse brain and then exposed mice to MPTP. Quantification of TH showed that loss of MEF2D function in mitochondria significantly accelerates the loss of TH signal in DA neurons in vivo (FIG. 7B). Over-expression of Mt2DVP16 or MtND6 in mitochondria partially attenuated MPTP-induced neuronal death (FIG. 7B). To probe the relevance of mitochondrial MEF2D to human disease, the levels of mitochondrial MEF2D and ND6 proteins in the brains of PD patients were assessed. Immunoblotting analysis revealed that the level of mitochondrial MEF2D is preferentially reduced in the brains of PD patients compared to matched controls (FIG. 7C), which correlates closely with a significant reduction in the level of ND6 protein.

### EXPERIMENTAL

### Purification of Mitochondria and Mitochondrial Subfractionation

Mitochondria were purified from brain tissue using discontinuous sucrose gradient method. Briefly, brain homogenate was made in ice-cold homo-buffer (0.32 M sucrose, 20 mM Tris-HCl, pH 7.4) and spun at 900×g, 4° C. for 10 min. The supernatant was transferred to another clean tube and spun at 10,000×g, 4° C. for 10 min. The resulted pellet from 10,000×g, enriched for mitochondria, was re-suspended in 2 ml homo-buffer, loaded on top of a sucrose gradient (1.2 M sucrose, 0.8 M sucrose, 0.32 M sucrose; 20 mM Tris-HCl, pH 7.4), and spun at 53,000×g, 4° C. for 2 hours. The white band at the interface between medium (0.8 M sucrose) and heavy (1.2 M sucrose) solutions was collected as highly purified mitochondria. Mitochondria from cultured cells were isolated using a kit (cat. no. 89874) from Pierce. Mitochondrial subfractionation was carried out as described by Hovius et al (40). Briefly, purified mitochondria (1 mg) were re-suspended in 500 μl of ice-cold buffer (10 μM KH2PO4, pH 7.4) and allowed to swell on ice for 20 min. Then 1 volume of iso-osmotic solution (32% sucrose, 30% glycerol, 10 mM MgCl2) was added and the mix was spun at 10,000×g at 4° C. for 10 min. The supernatant (S1) contained outer membrane and intermembrane space. The pellet (P1) was mitoplasts (matrix surrounded by intact inner membrane). P1 was re-suspended in 500 μl of ice-cold buffer (10 μM KH2PO4, pH 7.4) and allowed to swell on ice for 20 min. Then 1 volume of iso-osmotic solution (32% sucrose, 30% glycerol, 10 mM MgCl2) was added. S1 and the re-suspended P1 were spun at 15,000×g at 4° C. for 1 hr. The supernatant from S1 contained the intermembrane space and the pellet was the outer membrane. The supernatant from P1 contained the matrix and the pellet was the inner membrane.

### Immunofluorescence and Immunogold Electron Microscope

SN4741 cells or primary midbrain DA neurons were plated on glass slides in 24-well plates. Localizations of endogenous and exogenous MEF2D were visualized on a Zeiss LSM5 PASCAL confocal microscope. To visualize colocalization of MEF2D with the mitochondria, cells were stained with MitoTracker (Invitrogen) and anti-MEF2D (BD Bioscience) or anti-FLAG (Sigma) antibodies. Quantification of colocalization of signals was done using “Colocalization Analysis” program of software Image Pro (Media Cybernetics). Overlap coefficients of 50 cells in each group were collected. The overlap coefficient of the control group was set as 100%. For immunogold labeling of MEF2D, rat brains were fixed in 2% paraformaldehyde and 0.5% glutaraldehyde, embedded in London Resin White, and sectioned at 80 nm. Sections were incubated in 1:500 dilution of the monoclonal antibody to MEF2D. Goat anti-mouse IgG antibody conjugated to a 5 nm colloidal gold particle (Polysciences) was used as secondary antibody and photographed using a LEO EM-910 transmission electron microscope (LEO Electron Microscopy Inc.) at 80 kV. For the transmission electron microscope study on cultured cells, we diluted the anti-MEF2D antibody to 1:500 and goat anti-mouse IgG antibody conjugated to a 15 nm gold particle (Polysciences) was used as secondary antibody.

### In Vitro Import Assay

Mouse MEF2D cDNA was amplified by PCR using primers flanking the coding region. The T7 phage promoter was incorporated in the sense primer for in vitro transcription of the PCR fragment. In vitro transcription and translation were performed using the TNT T7 Quick Coupled Transcription/Translation System (Promega) in the presence of 20 μCi [³⁵S]methionine (Amersham). Fresh mitochondria prepared from 2×10⁷ SN4741 cells were suspended in incubation buffer to a final protein concentration of 2 mg/ml. The in vitro import assay was then carried out as described in Petruzzella et al., Genomics, 1998, 54:494-504. Samples were separated by SDS-PAGE. After fixation in 10% acetic acid/25% isopropanol, the gel was dried and exposed to HyBlot film (Denville) under −80° C.

### In Organello Transcription Assay

SN4741 cells were infected with Mt2D or Mt2Ddn lentiviruses for 24 hour. De novo transcription was measured in isolated mitochondria as described previously (46, 47). The mitochondrial fraction was suspended in transcription buffer containing 25 mM sucrose, 75 mM sorbitol, 100 mM KCl, 10 mM K₂HPO₄, 50 mM EDTA, 5 mM MgCl₂, 1 mM ADP, 10 mM glutamate, 2.5 mM malate, and 10 mM Tris-HCl (pH7.4), with 1 mg of BSA per ml. Mitochondria containing a total of 200 μg protein were incubated in 300 μl of the transcription buffer containing 0.1 mM Bio-11-UTP (Ambion) at 37° C. for 30 min. After the incubation, the mitochondria were pelleted, washed with PBS, solubilized in 100 ml of lysis buffer containing 50 mM Tris-HCl (pH 8.0), 20 mM NaCl, 1 mM EDTA, 1% SDS, and 20 mg of protease K (Gibco), and then incubated at room temperature for 15 min. The mitochondrial RNA was isolated by phenol extraction. Biotinylated mitochondrial RNA was isolated by streptavidin (Invitrogen) purification. Reverse transcription and ND6 real time PCR were done as described above. The primers for detection 16S RNA were: 5′-GTACCGCAAGGGAAAGATGAAAG-3′ (forward) and 5′-GGTAACCAGCTATCACCAAGCTC-3′ (reverse).

### Cell Culture

SN4741 cells were cultured at 33° C. with 5% CO2 in RF medium (DMEM supplemented with 10% FBS, 1% D-glucose, 1% penicillin-streptomycin, and 140 mM L-glutamine). When cells reached 70% confluence (usually 3 days), split it into 3 plates. Experiments were usually done in 12-well plate when cells reached 50-60 confluence. Primary mouse midbrain dopaminergic (DA) neurons were cultured as described by Son et al., J Neurosci, 1999, 19:10-20. Briefly, msencephalic SN regions from E13.5 embryos were surgically removed under sterile condition in L-15 media (Invitrogen). SN tissues were cut into small pieces, mechanically triturated in L-15 containing Trypsin-EDTA (final concentration, 0.1%), and incubated at 37° C. for 20 min. The reaction stopped by adding RF medium. The cells were pelleted and cultured in 12-well plate (5×105 cells/well) at 37° C. with 5% CO2 in RF medium. Experiments were done after DIV 7.

### Plasmids and Lentiviruses

Flag-tagged mouse MEF2D and ΔN30MEF2D were constructed by cloning PCR fragments into Nhe I/Not I sites of pcDNA3.1(+) (Invitrogen). Mitochondria-targeting MEF2D (Mt2D) and dominant-negative MEF2D (Mt2Ddn) were constructed by cloning mouse MEF2D 1-493aa or MEF2D 1-131aa into Age I/Not I sites of pDsRed2-Mito (Clontech), respectively. For lentivirus production, Mt2D and Mt2Ddn were subcloned into Xba I site of pFUGW using primers 5′-GATCCGCTAGCATGTCCGTCC-3′ (forward) and 5′-CGTCTAGACTAT TTATCGTCATCGTCTTTGTAG-3′ (reverse). Mt2DVP16 was constructed by fusion of TAD domain of VP16 with DNA-binding domain of MEF2D and mitochondria-targeting sequence (Mito) from pDsRed2-Mito (Clontech). MtND6 was constructed by fusion of Mito with ND6 sequence. All clones were confirmed by sequence. Lentiviruses were prepared by standard methodology.

### MPTP Mouse Model of Parkinson's Disease and Postmortem Human Brain Samples

The MPTP mouse model of PD was created as described by Bezard et al., Neurosci Lett, 1997, 234:47-50. Postmortem human brain samples were provided by the Brain Bank, Center for Neurodegenerative Disease, Emory University. The control (c) and PD (p) cases match in age (c, 70.4±5.3; p, 72.5±8.1), race (c and p, all Caucasian), sex (female/male: c, 6/7; p, 7/6), and postmortem interval (c, 7.8±3.4 hr; p, 8.3±3.2 hr). They are not diagnosed with other neurodegenerative diseases including Alzheimer's disease. All procedures performed in this study were approved by the Institutional Review Board (IRB) of Emory University.

### Mitochondrial Complex Activity Assays and Functional Assays

Mitochondrial complex I activity was evaluated initially by blue native-polyacrylamide gel electrophoresis (BN-PAGE) and in-gel activity staining. This assay measures total complex I activity. Rotenone sensitive complex I activity and activities of other mitochondrial complexes were further measured by methods described by Antoni et al., J Biol Chem, 1998, 273:14210-14217. Briefly, parallel assays were performed to measure complex I activity at 340 nm using 2,3-dimethoxy-5-methyl-6-n-decyl-1,4-benzoquinone (DB) (50 μM) as acceptor and NADH (0.8 mM) as donor, in 50 mM Tris (pH 8.0) buffer supplemented with 5 mg/ml BSA either without or with the addition of 4 μM rotenone, which gave total and rotenone insensitive complex I activities, respectively. The difference in value between the two provided a quantified measure of the rotenone-sensitive activity. Mitochondrial membrane potential was detected using a kit (cat. no. 280002) from Stratagene following the procedures provided by the manufacturer. The fluorescent dye JC-1 (5,5′,6,6′-tetrachloro-1,1′,3,3′-tetraethylbenzimidazolyl carbocyanine iodide) stains mitochondria red in a membrane potential dependent manner. Briefly, cells were seeded in 96-well plates at 4×104 cells/well with 100 μl culture medium and incubated at 33° C. with 5% CO2. After treatment, 10 μl/well of premixed JC-1 staining solution was added, and the cells were incubated for an additional 30 min under the same conditions. After thorough washes, 100 μl assay buffer was added to each well. Fluorescence signal was detected with excitation and emission at 520 nm and 590 nm, respectively, on a multi-well plate reader (Bio-Tek). Cellular ATP and H202 were measured by Bioluminescent Somatic Cell ATP Assay Kit (Sigma) and Hydrogen Peroxide Assay Kit (Cayman), respectively.

### Electrophoretic Mobility Shift Assay

Electrophoretic mobility shift assay (EMSA) was performed as described in Wang et al., Gastroenterology 2004, 127:1174-1188 (6). Briefly, 10 ng of purified proteins was used to incubate with 32P-ATP labeled specific probe or mutant probe on ice for 30 min. The reaction complexes were separated by a 5% non-denature polyacrylamide gel electrophoresis and visualized by autoradiography. The probe for the MEF2 site in ND6 was 5′-CTAAACCCCCATAAATAGGAGAAGGCTT-3′; for the mutant probe, the 3 nucleotides in bold were mutated to GGC.

### MEF2 Luciferase Reporter Assay

MEF2 luciferase reporter assay was done as described Wang et al., J Biol Chem, 2005, 280:16705-16713. Briefly, cells were transiently transfected with various constructs with MEF2 luciferase reporter plasmid (WT, reporter with wild type MEF2 DNA binding sites; mt, reporter with the MEF2 DNA binding sites mutated) using Lipofectimane 2000 (Invitrogen) following procedures provided by the manufacturer. A β-galactosidase expression plasmid was used to determine the efficiency in each transfection. The total amount of DNA for each transfection was kept constant by using control vectors. Cell lysates were analyzed for luciferase and β-galactosidase activity according to the manufacturer's instructions (Roche, cat. no. 11669893001).

### WST-1 Assay

WST-1 assay was done using a kit (cat. no. 11644807001) from Roche and following procedures provided by the manufacturer. WST-1 (4-[3-(4-Iodophenyl)-2-(4-nitrophenyl)-2H-5-tetrazolio]-1,3-benzene disulfonate) is a water-soluble tetrazolium salt whose cleavage by cellular enzymes correlates with cell viability. Cells were seeded in 96-well plates and treated as indicated. Than 10 μl/well of premixed WST-1 Cell Proliferation Reagent was added, and the cells were incubated for an additional 4 hr under the same conditions. Absorbance at 450 nm was measured on a multi-well plate reader (Bio-Tek).

1. A method of diagnosing a disease related to mitochondrial dysfunction in a subject comprising isolating a sample from a subject and exposing the sample to a reagent that indicates a level of a myocyte enhancer factor 2 (MEF2) isoform or its mitochondrial target gene ND6 in the sample, wherein the level of MEF2 isoform or ND6 is compared to a control level and wherein a level below a control level indicates presence of a disease in the subject.

2. The method of claim 1, wherein the MEF2 isoform is MEF2A-D.

3. The method of claim 1, wherein the disease is selected from Parkinson's disease, Alzheimer's disease Huntington's disease, amyotrophic lateral sclerosis, hereditary spastic paraplegia, cerebellar degenerations or any mitochondrial compromising disease.

4. The method of claim 1 wherein mitochondria of the sample are substantially isolated and wherein the level of MEF2 isoform and the level of ND6 in the mitochondria is compared to a control level of the same MEF2 isoform and ND6 in the sample prior to isolation.

5. The method of claim 1 wherein the sample is a blood sample of the subject.

6. A kit for diagnosing a disease in a subject comprising at least one reagent that indicates level of a MEF2 isoform or a level of ND6 and a reporter that allows measurement of the level.

7. The kit of claim 6, wherein the reagent is a nucleic acid primer or an antibody.

8. The kit of claim 6, wherein the reagent is an antibody.

9. An isolated chimeric protein comprising 1) a MEF2 isoform sequence and 2) a second mitochondrial-targeting signal.

10. The isolated chimeric protein of claim 9, wherein the MEF2 isoform is MEF2A, MEF2B, MEF2C, and/or MEF2D.

11. The isolated chimeric protein of claim 10, wherein the MEF2 sequence lacks a nuclear localization signaling sequence.

12. The isolated chimeric protein of claim 10, wherein the second mitochondrial-targeting signal is conjugated to the N-terminus of the MEF2 sequence.

13. The isolated chimeric protein of claim 10, wherein the second mitochondrial-targeting signal is the 30 N-terminal amino acid residues of MEF2D.

14. The isolated chimeric protein of claim 10, wherein the MEF2 sequence comprises a mutation.

15. A method of treating Parkinson's Disease or related disease comprising administering a pharmaceutical composition comprising a protein comprising a MEF2 sequence to a subject in need thereof.

16. The method of claim 15, wherein the protein is a chimeric protein comprising, 1) a MEF2 sequence and 2) a second mitochondrial-targeting signal, wherein said protein is lacking a nuclear localization signaling sequence.

17. The method of claim 15, wherein the MEF2 sequence is MEF2A-D.


SEQUENCE LISTING (3 sequences)

SEQ ID NO: 1 - protein, 521 aa, Homo sapiens
MGRKKIQIQRITDERNRQVTFTKRKFGLMKKAYELSVLCDCEIALIIFNHSNKLFQYASTDMDKVLLKYTEYNEPHESRTNADIIETLRKKGFNGCDSPEPDGEDSLEQSPLLEDKYRRASEELDGLFRRYGSTVPAPNFAMPVTVPVSNQSSLQFSNPSGSLVTPSLVTSSLTDPRLLSPQQPALQRNSVSPGLPQRPASAGAMLGGDLNSANGACPSPVGNGYVSARASPGLLPVANGNSLNKVIPAKSPPPPTHSTQLGAPSRKPDLRVITSQAGKGLMHHLTEDHLDLNNAQRLGVSQSTHSLTTPVVSVATPSLLSQGLPFSSMPTAYNTDYQLTSAELSSLPAFSSPGGLSLGNVTAWQQPQQPQQPQQPQPPQQQPPQPQQPQPQQPQQPQQPPQQQSHLVPVSLSNLIPGSPLPHVGAALTVTTHPHISIKSEPVSPSRERSPAPPPPAVFPAARPEPGDGLSSPAGGSYETGDRDDGRGDFGPTLGLLRPAPEPEAEGSAVKRMRLDTWTLK

SEQ ID NO: 2 - protein, 30 aa, Homo sapiens
MGRKKIQIQRITDERNRQVTFTKRKFGLMK

SEQ ID NO: 3 - protein, 507 aa, Homo sapiens
MGRKKIQIQRITDERNRQVTFTKRKFGLMKKAYELSVLCDCEIALIIFNHSNKLFQYASTDMDKVLLKYTEYNEPHESRTNADIIETLRKKGFNGCDSPEPDGEDSLEQSPLLEDKYRRASEELDGLFRRYGSSVPAPNFAMPVTVPVSNQSSMQFSNPSSSLVTPSLVTSSLTDPRLLSPQQPALQRNSVSPGLPQRPASAGAMLGGDLNSANGACPSPVGNGYVSARASPGLLPVANGNSLNKVIPAKSPPPPTHNTQLGAPSRKPDLRVITSQGGKGLMHHLNNAQRLGVSQSTHSLTTPVVSVATPSLLSQGLPFSSMPTAYNTDYQLPSAELSSLPAFSSPAGLALGNVTAWQQPQPPQQPQPPQPPQSQPQPPQPQPQQPPQQQPHLVPVSLSNLIPGSPLPHVGAALTVTTHPHISIKSEPVSPSRERSPAPPPPAVFPAARPEPGEGLSSPAGGSYETGDRDDGRGDFGPTLGLLRPAPEPEAEGSAVKRMRLDTWTLK